(19) United States
(12) Patent Application Publication
(10) Pub. No.: US 2021/0137468 A1
(43) Pub. Date: May 13, 2021

(54) PORTABLE HEALTH AND WELLNESS DEVICE

(71) Applicant: BHOGAR, LLC, Bloomfield Hills, MI (US)

(72) Inventors: Rakesh Babu Katragadda, Bloomfield Hills, MI (US); Saritha Vellanki, Bloomfield Hills, MI (US); Supreeth Harinath, Bangalore (IN)

(21) Appl. No.: 17/097,733

(22) Filed: Nov. 13, 2020

Related U.S. Application Data
(60) Provisional application No. 62/934,768, filed on Nov. 13, 2019.

(51) Int. Cl. A61B 5/00; A61B 5/0205; A61B 5/1171; H04W 4/38; G16H 40/63; G16H 40/67; H04L 29/06

(52) U.S. Cl. CPC A61B 5/7465; A61B 5/0022; A61B 5/02055; A61B 5/742; A61B 5/7475; A61B 5/021; A61B 5/1176; H04W 4/38; G16H 40/63; G16H 40/67; H04L 63/0428; A61B 5/002

(57) ABSTRACT

Described herein are portable diagnostic devices and related methods. A portable device may include: one or more probes configured to measure one or more health parameters, a strip sensing module configured to receive a test strip therein for measuring an analyte, a wireless communication module for transmitting data, a display, and a processor. Execution of instructions causes the processor to perform a method including: receiving a user input, on the display of the portable device, to initiate a remote health appointment with a healthcare provider; displaying the healthcare provider in a video, in real-time, on the display; acquiring the one or more health parameters; transmitting, in real-time to a remote computing device, the one or more health parameters to the healthcare provider during the remote health appointment; and receiving, in real-time on the portable device, a recommendation from the healthcare provider based on the one or more health parameters.

### CROSS-REFERENCE TO RELATED APPLICATIONS

This application claims the priority benefit of U.S. Provisional Patent Application Ser. No. 62/934,768, filed Nov. 13, 2019, which is herein incorporated by reference in its entirety.

### INCORPORATION BY REFERENCE

All publications and patent applications mentioned in this specification are herein incorporated by reference in their entirety, as if each individual publication or patent application was specifically and individually indicated to be incorporated by reference in its entirety.

### TECHNICAL FIELD

The invention relates generally to edge connected portable health and wellness kits that combine medical tests, patient management, localized machine learning or artificial intelligence, and telemedicine as a compelling healthcare kit.

### BACKGROUND

Currently, there are a number of solutions for doing point of care diagnostic tests and wellness parameters tests individually in separate devices. Some of these solutions attempt to ascertain the health of a person or the reason for his discomfort using very few tests, questionnaires, many independent devices, and the like, but these solutions fail to meet the needs of the industry. For example, the number of tests is not comprehensive; the operator needs to be a skilled technician; many independent and unconnected devices are used, each with their own user interface methods; and significant time is wasted performing each test serially. Other solutions attempt to do a basic set of tests or measure various biomarkers but depend on availability of internet connectivity to perform efficiently. Existing consumer devices, like wearables, need to be connected to the cloud and have access to the internet all the time in order to perform rudimentary machine learning or artificial intelligence tasks and in such cases, there are bandwidth bottlenecks and latency issues involved. Latency in medical technology is dangerous as that may lead to critical or emergent patients without proper medical equipment thus hampering critical care. In remote areas where there is no access to internet or in certain environments when the network connection is poor, many of the connected tasks cannot be performed. These solutions are similarly unable to meet the needs of the industry because availability of skilled technicians and constant high bandwidth internet is not feasible everywhere. Still other solutions seek to ascertain the nature and cause of discomfort by setting up helplines, doctor-on-call services, and dedicated health coaches/mentors for a fee, but these solutions also fail to meet industry needs. They do not have the backing of a clinical grade medical test report, clinically proven workflows leading to predictable outcomes, and continuous monitoring with minimal fuss and pain to the patient.

Accordingly, there is a need for improved edge enabled portable health and wellness assessment tools.

### SUMMARY

One aspect of the present disclosure is directed to a method of collecting one or more health parameters during a remote health appointment. In some embodiments, the method includes: providing a portable device configured to, substantially simultaneously, measure one or more health parameters and conduct a remote health appointment. In some embodiments, the portable device includes one or more probes configured to measure or collect one or more of: a blood pressure, a temperature, a blood oxygen saturation, an electrocardiogram, an image of an internal anatomical structure, or a combination thereof; a strip sensing module configured to receive a test strip therein for measuring an analyte; a wireless communication module for transmitting data to a remote computing device; a display; a processor communicatively coupled to the one or more probes, the strip sensing module, the wireless communication module, and the display; and a computer-readable medium having non-transitory, processor-executable instructions stored thereon. In some embodiments, execution of the instructions causes the processor to perform a method including: optionally receiving a biometric of a patient as input into the portable device; optionally verifying the patient of the portable device based on the biometric; receiving a user input, on the display of the portable device, to initiate a remote health appointment with a healthcare provider; displaying the healthcare provider in a video, in real-time, on the display; acquiring one or more health parameters using one or both of: the one or more probes or the strip sensing module of the portable device during the remote health appointment; transmitting, in real-time to a remote computing device, the one or more health parameters to the healthcare provider during the remote health appointment; and optionally receiving, in real-time on the portable device, a recommendation from the healthcare provider based on the one or more health parameters.

In some embodiments, the one or more health parameters are selected from the list consisting of: a blood pressure, a temperature, a blood oxygen saturation, an electrocardiogram, a blood glucose level, a uric acid level, a triglyceride level, a cholesterol level, an influenza antigen level, a coronavirus antigen level, an anti-influenza antibody level, an anti-coronavirus antibody level, or a combination thereof.

In some embodiments, transmitting, in real-time, further includes transmitting, in real-time, a patient health history to the healthcare provider during the remote health appointment.

In some embodiments, verifying the biometric of the patient further comprises receiving an image of the patient with an image sensor of the portable device to perform facial recognition of the image of the patient.

In some embodiments, the method performed by the processor further includes automatically detecting which of the one or more probes are connected to the portable device before acquiring the one or more health parameters.

In some embodiments, the method performed by the processor further includes transmitting a remote user input, from the healthcare provider, from the remote computing device to the portable device to initiate the acquisition of the one or more health parameters using the portable device.

In some embodiments, the method performed by the processor further includes transmitting, in real-time, a prescription to a pharmacy for the patient based on the remote health appointment or the acquired one or more health parameters.

In some embodiments, the method performed by the processor further includes encrypting the one or more health parameters before transmission.

Another aspect of the disclosure is directed to a portable device of collecting one or more health parameters during a remote health appointment. In some embodiments, the device includes: one or more probes configured to measure or collect one or more of: a blood pressure, a temperature, a blood oxygen saturation, an electrocardiogram, an image of an internal anatomical structure, or a combination thereof; a strip sensing module configured to receive a test strip therein for measuring an analyte; a wireless communication module for transmitting data to a remote computing device; a display; a processor communicatively coupled to the one or more probes, the strip sensing module, the wireless communication module, and the display; and a computer-readable medium having non-transitory, processor-executable instructions stored thereon.

In some embodiments, execution of the instructions causes the processor to perform a method including: optionally receiving a biometric of a patient as input into the portable device; optionally verifying the patient of the portable device based on the biometric; receiving a user input, on the display of the portable device, to initiate a remote health appointment with a healthcare provider; displaying the healthcare provider in a video, in real-time, on the display; acquiring one or more health parameters using one or both of: the one or more probes or the strip sensing module of the portable device during the remote health appointment, wherein the one or more health parameters are selected from the list consisting of: a blood pressure, a temperature, a blood oxygen saturation, an electrocardiogram, a blood glucose level, a uric acid level, a triglyceride level, a cholesterol level, an influenza antigen level, a coronavirus antigen level, an anti-influenza antibody level, an anti-coronavirus antibody level, or a combination thereof; transmitting, in real-time to a remote computing device, the one or more health parameters to the healthcare provider during the remote health appointment; and receiving, in real-time on the portable device, a recommendation from the healthcare provider based on the one or more health parameters.

In some embodiments, the device further includes a housing defining one or more ports configured to receive one or more connectors for the one or more probes therein.

In some embodiments, the wireless communication module includes one or more of: a Bluetooth module, a Wi-Fi module, or a 3G/4G modem.

In some embodiments, the remote computing device comprises a healthcare provider computing device. In other embodiments, the remote computing device comprises a server or a cloud-based database.

In some embodiments, the device further includes an image sensor configured to take an image of the patient as the biometric of the patient, such that the processor is configured to perform facial recognition on the image of the patient.

In some embodiments, the device further includes a speaker and microphone to facilitate communication during the remote health appointment.

In some embodiments, the device further includes a wireless scale communicatively coupled to the processor, such that the wireless scale is configured to transmit a weight and a body composition assessment of the patient to the processor of the portable device.

In some embodiments, the display is a touch-enabled interactive configurable display.

In some embodiments, the processor is a medical data configurable processor.

In some embodiments, the one or more probes include one or both of an ultrasound probe or a scope for taking the image of the internal anatomical structure.

Another aspect of the present disclosure is directed to a method of collecting one or more health parameters during a remote health appointment. The method includes providing a portable device configured to, substantially simultaneously, measure one or more health parameters and conduct a remote health appointment. In some embodiments, the portable device includes: one or more probes configured to measure or collect one or more of: a vital parameter, a blood parameter, a viral parameter, or an image of an internal anatomical structure, or a combination thereof blood pressure; a wireless communication module for transmitting data to a remote computing device; a display; a processor communicatively coupled to the one or more probes, the strip sensing module, the wireless communication module, and the display; and a computer-readable medium having non-transitory, processor-executable instructions stored thereon. In some embodiments, execution of the instructions causes the processor to perform a method including: optionally receiving a biometric of a patient as input into the portable device; optionally verifying the patient of the portable device based on the biometric; receiving a user input, on the display of the portable device, to initiate a remote health appointment with a healthcare provider; displaying the healthcare provider in a video, in real-time, on the display; acquiring the one or more health parameters using one or both of: the one or more probes or the strip sensing module of the portable device during the remote health appointment; transmitting, in real-time to a remote computing device, the one or more health parameters to the healthcare provider during the remote health appointment; and receiving, in real-time on the portable device, a recommendation from the healthcare provider based on the one or more health parameters.

In some embodiments, acquiring further includes: receiving one or more signals from the one or more probes, and extracting the one or more health parameters from the one or more signals locally on the portable device using edge computing methods.

In some embodiments, the viral parameter includes one or more of: an influenza antigen level, a coronavirus antigen level, an anti-influenza antibody level, an anti-coronavirus antibody level, or a combination thereof.

In some embodiments, the vital parameter includes one or more of: a temperature, a blood oxygen saturation, an electrocardiogram, a weight, a blood pressure, or a combination thereof.

In some embodiments, the blood parameter includes one or more of: a blood glucose level, a uric acid level, a triglyceride level, a cholesterol level, or a combination thereof.

### BRIEF DESCRIPTION OF THE DRAWINGS

The foregoing is a summary, and thus, necessarily limited in detail. The above-mentioned aspects, as well as other aspects, features, and advantages of the present technology are described below in connection with various embodiments, with reference made to the accompanying drawings.

FIG. 1 illustrates a schematic of one embodiment of a portable diagnostic kit.

FIG. 2 illustrates a perspective view of one embodiment of a portable diagnostic kit.

FIG. 3 illustrates another perspective view of one embodiment of a portable diagnostic kit.

FIGS. 4A-4B illustrate a first side or right side and a second or left side, respectively, of one embodiment of a portable diagnostic kit.

FIG. 5 illustrates an interior view of a front portion of one embodiment of a portable diagnostic kit.

FIG. 6 illustrates a bottom view of one embodiment of a portable diagnostic kit.

FIG. 7 illustrates an interior view of a back portion of one embodiment of a portable diagnostic kit.

FIG. 8 illustrates an external view of a back portion of one embodiment of a portable diagnostic kit.

FIG. 9 illustrates an external view of a front portion of one embodiment of a portable diagnostic kit.

FIG. 10 illustrates a schematic of a method of initialization or authentication of one embodiment of a portable diagnostic kit.

FIG. 11 illustrates a schematic of a method of profile creation of one embodiment of a portable diagnostic kit.

FIG. 12 illustrates a schematic of a method of customizing, adjusting, or setting system settings of one embodiment of a portable diagnostic kit.

FIG. 13 illustrates a schematic of a method of customizing, adjusting, or setting options of one embodiment of a portable diagnostic kit.

FIG. 14 illustrates a schematic of a method of recording ECG of one embodiment of a portable diagnostic kit.

FIG. 15 illustrates a schematic of a method of measuring blood pressure and/or pulse rate of one embodiment of a portable diagnostic kit.

FIG. 16 illustrates a schematic of a method of measuring an analyte of one embodiment of a portable diagnostic kit.

FIG. 17 illustrates a schematic of a method of measuring one or more of: a SpO2, a temperature, an ECG, and/or a weight of one embodiment of a portable diagnostic kit.

FIG. 18 illustrates a schematic of a method of measuring an ECG of one embodiment of a portable diagnostic kit.

FIG. 19 illustrates a schematic of a method of assessing body composition of one embodiment of a portable diagnostic kit.

FIG. 20 illustrates a schematic of a method of collecting one or more health parameters during a remote health appointment.

The illustrated embodiments are merely examples and are not intended to limit the disclosure. The schematics are drawn to illustrate features and concepts and are not necessarily drawn to scale.

### DETAILED DESCRIPTION

The foregoing is a summary, and thus, necessarily limited in detail. The above-mentioned aspects, as well as other aspects, features, and advantages of the present technology will now be described in connection with various embodiments. The inclusion of the following embodiments is not intended to limit the disclosure to these embodiments, but rather to enable any person skilled in the art to make and use the contemplated invention(s). Other embodiments may be utilized, and modifications may be made without departing from the spirit or scope of the subject matter presented herein. Aspects of the disclosure, as described and illustrated herein, can be arranged, combined, modified, and designed in a variety of different formulations, all of which are explicitly contemplated and form part of this disclosure.

It would be desirable to have a device that can measure health and wellness biomarkers quickly, accurately, and with minimal pain to the patient. Further, it would be desirable to have a device that can perform health and wellness assessments at the patient's home at his/her convenience and comfort. Furthermore, it would also be desirable to have a device that can cover almost all the health and wellness biomarkers that are typically done at a diagnostic lab but without the pain of going through drawing of blood from a vein. Still further, it would be desirable to have a device that can quickly communicate the test results to expert clinicians instantaneously and obtain consultation results without the need for a physical visit to a doctor for consultation. Therefore, there currently exists a need in the industry for a device and associated method that does most of the health and wellness biomarkers at the patient's house/convenience with minimal pain, maximum efficiency, and good accuracy. Current devices are incapable of meetings these needs. For example, currently available devices suffer from several technical problems: they are large and cumbersome; include passive display systems making it difficult for untrained users to effectively operate; utilize multiple disparate systems for obtaining all the test results (e.g., biomarkers, vitals, wellness, health, etc.) that may be needed; send data to a server for analysis leading, at least to lag time and data security concerns; and are often not personalized for a specific healthcare provider (e.g., personalized alarms or alerts). As such, technical solutions are needed to solve the aforementioned technical problems. Provided herein are such technical solutions.

Further, the disclosed devices and methods are unique when compared with other known devices and solutions because they provide: (1) comprehensive tests of a plurality of biomarkers (e.g., greater than 25, greater than 30, about 5 to about 30, etc.) covering most of the basic health and wellness tests prescribed by doctors; (2) kight weight, portable, and battery operated (e.g., fits into a backpack); (3) single uniform workflow with on-screen help for all tests; and (4) device operation requires low skill and doesn't require a trained paramedic or a nurse.

Further, the disclosed device is unique in that it is structurally different from other known devices or solutions. More specifically, the device is unique due to the presence of: (1) a plurality of biomarker tests in a form factor box; (2) combines vital sign monitoring, BCA, computing device, and a strip sensing module (e.g., biochemistry analyzer) into a single machine and single work-flow; (3) clinically proven workflows for prognosis and diagnosis; and (4) instantaneous physical printed reports for record keeping.

Similarly, the associated methods are unique in that: (1) all the tests, workflows, patient profile entry, trend recall, test performing and results are all integrated into one single device; (2) video remote consultation with a healthcare provider requires no additional equipment like laptop or tablet but can be done from within the user interface of the device; and/or (3) prescriptions and medicines are managed on a per patient per appointment basis until treatment completion. Similarly, the disclosed method is unique when compared with other known processes and solutions in that: (1) it performs a plurality of biomarker tests on a single device (more than any other point of care device); (2) has clinical grade results within 15 minutes rather than several hours; (3) has ability to add more tests easily without incurring significant additional costs and re-training of operator; and (4) weighs less than 3.0 kilograms.

Furthermore, the process associated with the aforementioned device is likewise unique. More specifically, the disclosed process owes its uniqueness to the fact that it: (1) performs different tests in the same uniform way to make it possible for low skilled person to operate the device; (2) ability to view past visits data and trend analytics on the point of care device itself; and (3) ability to do video consultation in a diagnostic kit in addition to doing the biomarker tests.

Such a device would facilitate a remote consultation with a healthcare provider; substantially simultaneously acquire needed vital, viral, and/or blood tests; and generate recommendations (e.g., prescriptions, treatments, specialists, etc.) within a short time period and all in one portable battery-operated device that, for example, can fit into a backpack. The portable medical diagnostic device may automatically generate electronic prescriptions based on biomarkers within minutes of completing the tests. The portable medical diagnostic device can instantaneously generate a health score based on a large sampling of biomarkers (e.g., a plurality of biomarkers, more than one biomarker, one or more biomarkers, etc.) measured and patient profile data, at the point-of-care. The health score may be weighted based on a collection of parameters, demographic data or population data, and/or past patient data.

In addition to telemedicine or remote medicine, the devices and methods described herein may also be used in step-down ICUs and during bedside monitoring in hospitals and clinics. For example, the devices and methods described herein use a combination of non-invasive and invasive methods for monitoring patient's hemodynamic, vital, and blood parameters.

As used herein, a portable device, portable diagnostic device, a portable diagnostic kit, a health and wellness diagnostic kit, a diagnostic kit, a wellness kit, a diagnostic device, a wellness device, etc. may be used interchangeably. In some embodiments, a portable diagnostic kit (PDK) may be about 4 inches to about 8 inches in height, about 8 inches to about 12 inches wide, and about 2 inches to about 6 inches deep, such that it is easily transportable, usable, and configurable on any surface.

In some embodiments, a PDK may be sized and configured such that when a patient or user schedules a remote health appointment, a PDK may be shipped or transported to the patient for its meeting or a PDK may be picked up at a local facility, for example a hospital or clinic. Further, the PDK may be configured such that very little to no training is required to use the PDK. For example, a PDK may display instructions, feedback, and/or recommendations to the patient to maximize a quality of each test.

Disclosed herein is a portable battery operated point of care health and wellness diagnostic kit, which may include one or more of the following components: a power source (e.g., rechargeable battery); a display (e.g., touchscreen interface); one or more probes for measuring or collecting one or more of the following: non-invasive blood pressure (NIBP), oxygen saturation (SpO2), electrocardiogram (ECG), temperature, an image of an internal anatomical structure (e.g., ultrasound probe, scope, etc.); one or more connected devices to measure a height, weight, and/or body composition assessment (BCA); one or more test strips (e.g., for receiving one or more drops of blood each) for measuring an analyte (a glucose, a uric acid, a triglyceride, a cholesterol, viral particle, antibody, antigen, etc.) in a bodily fluid; and/or patient preparation devices (e.g., lancing device for finger prick; alcohol swabs, cotton wool, etc.). One or more of these components are connected to the PDK (e.g., via a housing) via specific physical connector ports on the device, or wirelessly connected through Bluetooth or Wi-Fi. For example, NIBP, ECG, SPO2, temperature, and height may be physical connected via connectors on the device, while weight and body wellness parameters may be wirelessly connected (e.g., via Bluetooth, near-field communication, etc.). The device may connect to the internet data servers using in-built Wi-Fi/GPRS/GSM/CDMA modem.

In some embodiments, the PDK is edge-computing enabled (e.g., via an edge-computing enabled processor) such that data acquisition and at least some of the analysis occur locally on the PDK, as opposed to sending the data to a server or remote computing device for analysis. Such functionality allows a remote health appointment to be conducted substantially simultaneously during data acquisition (e.g., vitals, blood work, etc.) and processing.

The device may also be configured to perform one or more of the following: detect anomalies in one or more sensed health parameters (e.g., arrhythmia conditions in an ECG signal); display trends and/or efficacy plots for all the sensed health parameters to indicate health improvements or changes in health relative to past appointments; print reports instantly with an optionally attached thermal printer; and/or connect to other biomarker meters for cell counter, electrolytes, and urine analysis, etc. Similarly, the associated method may also include one or more of the following steps: selecting and initiating one or more tests using one or more connected probes or modules, waiting for results, and proceeding with clinical workflows just like for other health parameters tests. Such workflow may occur substantially simultaneously with a remote health appointment all on the same device.

In some embodiments, the PDK may also be configured to optionally connect (e.g., wired or wireless connection) to scales with built-in BCA parameters and store it against the patient profile. In some embodiments, the PDK is configured to store patient tests, profile, trends, and past appointment data locally on the device and retrieve it instantaneously on the display for usage by patients and clinicians, for example during a remote health appointment.

This disclosure will now provide a more detailed and specific description that will refer to the accompanying drawings. The drawings and specific descriptions of the drawings, as well as any specific or alternative embodiments discussed, are intended to be read in conjunction with the entirety of this disclosure. The portable health and wellness kit that combines medical tests, patient management, and remote medicine as a compelling healthcare kit may, however, be embodied in many different forms and should not be construed as being limited to the embodiments set forth herein. Rather, these embodiments are provided by way of illustration only and so that this disclosure will be thorough, complete and fully convey understanding to those skilled in the art.

In one embodiment, the device includes one or more of the following components: a power source (e.g., connectable to wall outlet or rechargeable battery); a display; a wireless communication module including one or more of: a Bluetooth module, a Wi-Fi module, or a 3G/4G modem; one or more probes for measuring or collecting blood pressure, SpO2, ECG, heart rate, respiratory rate, forced vital capacity (e.g., via spirometer), forced expiratory volume (e.g., via spirometer), temperature, an analyte, an image (e.g., via ultrasound, scope, etc.) of an internal anatomical structure (e.g., kidneys, bladder, ear, nose, throat, etc.); etc.; optionally one or more connected (wired or wirelessly) devices (e.g., digital stethoscope, height, weight, BCA; or a strip sensing module (e.g., test strips). In some embodiments, an optional custom-built backpack accommodates all the probes and accessories in separate cushioned compartments within the backpack. The in-built battery can be charged via the provided wall plug power adaptor. The one or more probes are connected to the device, for example via a housing defining one or more ports, via specific physical connector ports on the device clearly color coded and matched with the probes for easy identification, or wirelessly connected through Bluetooth or Wi-Fi. For example, NIBP, ECG, SpO2, temperature, height, and spirometer may be physically connected via connectors on the device. Further for example, weight, body composition parameters, and digital stethoscope may be wirelessly connected, for example over Bluetooth. The device connects to the internet data servers using in-built GPRS/GSM/CDMA modem.

In some embodiments, the one or more probes are configured to collect or measure one or more of: a vital parameter, a blood parameter, a viral parameter, a hemodynamic parameter, and/or an image of an internal anatomical structure (e.g., via ultrasound, scope, etc.). For example, the vital parameter may include, but not be limited to: a temperature, a blood oxygen saturation, an electrocardiogram, a weight, a BCA, a blood pressure, a heart rate, a respiratory rate, a forced vital capacity, a forced expiratory volume, etc. A viral parameter may include, but not be limited to, one or more of: a viral particle load, a viral antigen level, an anti-viral antibody level, an influenza antigen level, a coronavirus antigen level, an anti-influenza antibody level, an anti-coronavirus antibody level, etc. A blood parameter includes, but is not limited to: blood glucose, uric acid, triglyceride, cholesterol, hematocrit, hemoglobin, calcium, lipoprotein, vitamins, iron, blood urea nitrogen, creatinine, albumin, CO2, sodium, potassium, chloride, triiodothyronine (T3), thyroxine (T4), thyroid-stimulating hormone (TSH), calcitonin, CPK isoenzymes, heart enzymes, liver enzymes, etc. A hemodynamic parameter may include, but not be limited to: ECG (e.g., ST-Segment and arrhythmia; respiration; NIBP (e.g., systolic pressure, diastolic pressure, and mean pressure); temperature; and SpO2.

As such, the PDK is a portable patient monitor that measures various parameters of the patient and is used in clinical monitoring of adults and pediatrics. It uses both invasive and non-invasive methods for monitoring patient's parameters such as blood glucose, ECG, blood pressure, SpO2, and temperature. In some embodiments, the PDK also features an image sensor to take one or more images of the patient.

In some embodiments, a PDK optionally includes a biometric sensor for acquiring a biometric of the user and verifying the user of the device based on the biometric. For example, a biometric sensor may include an image sensor, such that an image of a user is taken so that facial recognition processing can verify the user of the device. Alternatively, or additionally, a biometric sensor may include a contact sensor for sensing a fingerprint of a user and verifying the user based on the fingerprint. Alternatively, or additionally, a biometric sensor may include low-energy infrared light for performing a retinal scan for user verification. Alternatively, or additionally, user verification may be performed using one or more encrypted digital keys, 2-factor authentication, or the like. In some embodiments, a biometric scan or input is verified using artificial intelligence or machine learning algorithms that use pattern recognition to ensure the authenticity of the patient; and upon confirming the identity enable health parameter assessment, use of the PDK, a remote appointment with a healthcare provider, or a combination thereof.

In some embodiments, a PDK is optionally wirelessly connected to a remote computing device. The remote computing device may be a server or cloud computing device or a healthcare provider computing device. For example, the healthcare computing device may be used by a healthcare provider during a remote health appointment.

In some embodiments, a PDK further includes a speaker and/or microphone to facilitate communication with a healthcare provider during a remote health appointment. Further, the speaker may be further used to audibly alert a user of the PDK, for example that a test is running or finished, a limit has been reached, a prescription has been sent or is ready, a message has been received from a healthcare provider, a reminder that a remote health appointment is occurring, etc. Further, the microphone may be used so that a patient may use speech to text to interact with the PDK, use speech to operate various aspects of the device, and/or interact with a healthcare provider during a remote health appointment.

In some embodiments, performing the method associated with the disclosed device includes: turning on the device, creating a patient profile with bibliographic data (e.g., name, address, mobile number, ID proof details, photo, pre-existing ailments, complaints, allergies, etc.); selecting one or more tests to be performed using the user interface; following the preparation steps indicated on-screen by connecting the probe to or onto the patient and/or collecting a bodily fluid sample for analysis via the strip sensing module; initiating the test by confirmation; and waiting for results. In some embodiments, the method includes sharing the results with a healthcare provider by short message service (SMS), email, or other digital communication method, and setting up a remote consultation or appointment with the healthcare provider, for example simultaneously as tests results are acquired. For example, the portable diagnostic device can immediately perform a remote consultation video call with a healthcare provider based on the severity of the test results. In some embodiments, the method includes receiving a recommendation (e.g., prognosis, diagnosis, therapy, prescription, etc.) from the clinician electronically, and/or establishing prescription delivery after obtaining consent from the patient. In some embodiments, the method includes closing the appointment and informing the patient how to access his reports online anytime with secure login/password credentials provided. The portable diagnostic device can automatically arrange for delivery of medicines based on test results measured at the point-of-care within, for example 90 minutes.

The portable diagnostic device with touch interface allows patient profiles to be created, parameters measured, and stored for future retrieval and results to be shared with clinicians with the touch of a button, as will described in greater detail below.

Referring now to FIG. 1, which illustrates a hardware block diagram of one embodiment of a portable diagnostic device, with its subsystems and peripherals identified.

The SBC (Single Board Computer) or processor B19 runs a custom-built real-time kernel. The custom-built real-time kernel receives input from various medical devices connected to the portable device and combines them into one unifying medical output. Processor B19 connects to various peripherals to collect test result data of different patient health and wellness parameters. Processor B19 analyzes the data, packages the data streams, and stores locally on solid-state nonvolatile memory B41. Such analysis may be performed locally using edge-computing methods. All the patient records are first stored on local memory B41 and then pushed to internet database B35 (e.g., cloud, server, etc.) over Wi-Fi (Wi-Fi module B33 connected to internet through internet gateway B34) or over 3G/4G modem B31, which in turn is connected to internet database B35. Processor B19 may be an in-built medical processor configured to take multiple complex medical signals and condense them into one simple machine-readable format and/or perform programmable computation of a medical condition.

Each patient profile and associated records can be retrieved on the display B10 by entering the ID of the patient or other patient unique credentials. The patient data is entered using the touchscreen interface on display B10. User input handler B11 allows for having different types of user input (e.g., speech, text, audio images, ECG/EKG waveforms, etc.), which in the base model is through a touchscreen interface on display B10. Although touchscreens are specifically described herein, one of skill in the art will appreciate that employing other display types will not substantively depart from the scope of this disclosure.

Patient records B36 are stored on an online cloud server, which is accessed by a simple workflow running on a browser as webpages, for example. Patient records B36 can also be accessed by patients for viewing their own patient records and past appointment or test data. Patients can also share their medical records with their own pre-authorized healthcare providers for review.

In some embodiments, microphone B37, speaker B38 and/or camera B14 allow the user to initiate a video call with a clinician. This facilitates remote consultation without the need for a physical visit or meeting. Such remote consultation or appointment on the portable diagnostic device can occur substantially simultaneously as various vital, viral, and/or blood tests performed using the portable diagnostic device.

Bluetooth module B12 is connected to the motherboard SBC or processor B19 on a wired line, which facilitates connecting to a BCA machine and weight machine or other hardware wirelessly. In some embodiments, it also connects to other devices like a adigital stethoscope, a spirometer, etc. All these are grouped as external devices B13.

Although wireless connections B39 and wired connections B40 are shown in FIG. 1, one of skill in the art will appreciate that wireless connections can be wired, and wired connections can be made wireless without departing from the scope of this disclosure.

Power management B32 manages all the different power sources and generates appropriate voltage and current for proper operation of all the sub-systems. It also does power saving functionalities by intelligently controlling power to signal conditioner/feature extractor B15, signal conditioner/feature extractor B21, power source B30, display B10, Bluetooth module B12, Wi-Fi module B33, and 3G/4G modem B31. Fuse B29 protects the sensitive hardware in case of high current surges from power input control B28 when external power sources are used.

Power source B30 (e.g., rechargeable battery pack) powers the entire system when there is no external power source or when an external power source fails (e.g., outage, rural, etc.). This is especially useful when availability of external power is not always guaranteed.

Strip sensor module B22 senses a presence of an electronic strip and activates signal conditioner/feature extractor B21 to do further processing. Signal conditioner/feature extractor B21, upon activation by strip sensor B22, identifies the type of the electronic strip (e.g., glucose B23, uric acid B25, triglyceride B26, or cholesterol B27) and informs the SBC B19 to take further action. For example, the signal conditioner/feature extractor B21 reads the electronic strip (e.g., barcode, QR code, NFC, tag, etc.), send the strip type to the processor, which initiates a method of detecting and analyzing the analyte of the strip. For example, a method similar to that of FIG. 16 may be performed. SBC B19 outputs appropriate messages and guiding inputs to the user on display B10 for completion of the tests and obtain results.

As a non-limiting examples: glucose strip B23 is an electronic strip for doing finger prick-based blood glucose testing; uric acid strip B25 is an electronic strip for doing finger prick-based uric acid testing; triglyceride test strip B26 is an electronic strip for doing finger prick-based triglycerides testing; and cholesterol test strip B27 is an electronic strip for doing finger prick-based total cholesterol testing. As described elsewhere herein, a plurality of other analytes are contemplated for testing via the disclosed devices and methods.

Signal conditioner/feature extractor B15 is the hardware used for sensing connection of patient side probes for performing health parameter tests. Signal conditioner/feature extractor B15 acquires the analog signals from each of the probes, for example, blood pressure cuff B16, temperature probe B17, pulse oximeter B18, and ECG probe B20; processes them to extract features; and transmits the same to SBC B19, for example over a wired or wireless communication line. SBC B19 does further analysis to generate the test results and waveforms, which are, then stored on the local nonvolatile memory B41 as patient records B36. Each probe or connector may be color coded, uniquely shaped, or otherwise uniquely tagges to avoid confusion while connecting.

One embodiment of a portable diagnostic device or kit is shown in FIG. 2. A portable battery-operated device can measure or collect health parameters, hemodynamic parameters, respiratory parameters, blood parameters, vital parameters, viral parameters, analytes, wellness parameters, images of internal anatomical structures, etc. as described elsewhere herein.

Now referring to at least FIG. 3, which shows the different connectors and interfaces for operating the PDK. The device is turned ON with the power switch 101. An external power source is connected to the device via connector 111. When there is presence of power via connector 111, and if the switch 101 is turned to ON position, the device is powered up automatically. The display 110 will be activated at power ON and displays the HOME screen to start interacting with the user. With switch 101 in ON position, the internal battery may be automatically charged. In some embodiments, the power switch 101 includes a built-in light (e.g., LED) to indicate availability of the external power when turned to the ON position.

A housing of the PDK defines one or more ports for connecting one or more probes to the PDK. The probes, ports, and/or connectors may be color coded or otherwise labeled to ensure proper connection. The various ports are configured to connect one or more probes for measuring or collecting different parameters of the patient. For example, temperature port 102 is for connecting a temperature probe; blood pressure port 103 if for connecting an NIBP (noninvasive Blood pressure) Cuff; pulse oximetry port 104 is for connecting an SpO2 finger cuff; and ECG5 port 106 for connecting a 5-channel ECG probe. Strip port 107 in the strip sensing module is configured to receive different electronically coded strips (e.g., barcode, near-filed communication tag, chip, other tag, etc.) for measuring different blood parameters or analytes. ECG12 port 109 is for connecting a 12-channel ECG probe. Air vent ports 105 are configured to maintain temperature inside the device within operating limits.

In some embodiments, a portable health and wellness device includes a housing comprising a front portion or sidewall 108 and a back or rear portion or sidewall 112. Front portion or sidewall includes display 110. The front portion and back portion couple together to form the portable device. All the electronics, cables, batteries and the like, are housed inside the housing. Front portion 108 and back portion 112 are mechanically held together through standard screw/thread assembly or the like.

Now referring to FIGS. 4A-4B, which show indentations or ledges 148, 149 formed on a first side (or right side when facing screen) and second side (or left side when facing screen), respectively, on the back portion 112 of the housing. Ledge 148 may be opposite ledge 149, for example on a left and right side, respectively of the device. Ledges 148, 149 may function as handles or grips during transport or use or for positioning the portable device on a surface.

Now referring to FIG. 5, which illustrates an interior view of a front portion of a portable diagnostic kit, with the back portion 112 of the housing removed. Threaded bosses 127, 128 and 147 are on the corners of the front portion 108 of the housing through which screws are installed from the back portion 112 of the housing to mechanically hold the housing together, front and back portions, as one single unit.

Power source 129 (e.g., rechargeable battery pack) powers the device when there is no external power input to the device. When there is external power input, power source 129 continuously charges until it is fully charged. NIBP inflation motor 130, when actuated, inflated the NIBP cuff.

Hardware board 140 performs analog signal conditioning and feature extraction for the measured parameters, such as temperature, SpO2, ECG, and NIBP. Hardware board 140 is connected to SBC or processor 113 through a dedicated wired connection for information exchange and/or command control operations.

Switch 141 is the panel-mounted power ON control switch for the entire device. Port 142 is the panel mounted power jack for receiving external power input.

In some embodiments, a portable device or portable diagnostic kit may operate optimally between about 5° C. and about 40° C. at a humidity, during operation, of greater than or equal to about 8% and, during non-operating time, of less than or equal to about 95%.

In some embodiments, a power supply for the portable diagnostic kit may require about 100 V to about 240 V, about 60 Hz, and/or about 5.0 Volt DC. Voltage may fluctuate about +/−10% without damage to the device. In some embodiments, the device has a maximum power consumption of about 25 watts and an over current protection of about 5 A.

Temperature connector 143 is the panel-mounted connector for receiving a temperature probe. Temperature connector 143 is connected to hardware board 140 through a dedicated wired cable. The temperature probe may be a resistance temperature detector, a thermocouple, negative temperature coefficient thermistor, or a semiconductor-based sensor. The temperature probe may be configured to measure temperatures ranging from about 25° C. to about 45° C. with +/−0.2° C. accuracy in less or equal to about 150 seconds.

Blood pressure connector 144 is the panel-mounted connector for receiving a NIBP cuff. Blood pressure connector 144 is connected to hardware board 140 through a dedicated wired cable. In some embodiments, the blood pressure cuff is configured to measure a systolic pressure range of about 40 mmHg to about 270 mmHg for adults and about 40 mmHg to about 200 mmHg for pediatrics. The blood pressure cuff may be further configured to measure a diastolic pressure range between about 10 mmHg and about 210 mmHg for adults and between about 10 mmHg and about 150 mmHg for pediatrics. The blood pressure may apply pneumatic pressure during measurement ranging from about 0 mmHg to about 300 mmHg with +/−3 mmHg accuracy. The blood pressure cuff may be further configured to measure a mean arterial pressure between about 20 mmHg to about 230 mmHg for adults and between about 20 mmHg and about 165 mmHg for pediatrics. A blood pressure accuracy measured by the system may have a mean error of less than or equal to about +/−5 mmHg with a standard deviation of less than or equal to about 8 mmHg Pulse rate may also, or alternatively, be measured with the blood pressure cuff, such that rates between about 40 beats per minute (bpm) and about 240 bpm with +/−2% accuracy may be measured. A blood pressure of a user or patient may be acquired in about 30 seconds or less for an adult and about 20 seconds or less for pediatrics. Over pressure protection has a limit of about 320+/−10 mmHg by dual transducers. In some embodiments, the portable diagnostic device detects a stable point in SpO2 and pulse rate simultaneously to improve the accuracy of NIBP measurement.

Pulse oximeter connector 145 is the panel-mounted connector for receiving an SpO2 probe. Pulse oximeter connector 145 is connected to hardware board 140 through a dedicated wired cable. The SpO2 probe may be reflectance-based or transmission based. A measurement range of the SpO2 probe me be about 35% to about 99%. The root-mean-square accuracy of the measurement may be less than or equal to about 3% in measurements ranging from about 70% to about 100%. In some embodiments, pulse rate may also be measured with an SpO2 probe. The SpO2 probe may be configured to measure a pulse between about 30 beats per minute (bpm) and bout 240 bpm with about +/−2% bpm accuracy. The pulse rate may update about every 5-10 pulse beats, about every 6-10 pulse beats, about every 8 pulse beats, about every 7-9 pulse beats, etc. In some low perfusion settings, the SpO2 probe may still meet accuracy requirements even when a pulse modulation ratio is greater than equal to about 0.4%.

ECG connector 146 is the panel-mounted connector for receiving ECG probe. ECG connector 146 is connected to hardware board 140 through a dedicated wired cable. The ECG may have a measurement range of about +/−0.5 mVp to about +/−5 mVp. A heart rate display range may be between about 20 bpm and about 300 bpm with an accuracy of about +/−2% or about +/−2 bpm, whichever is greater. An ECG may be configured with an alarm delay time of less than or equal to about 12 seconds and an input noise level for the amplifier of less than or equal to about 30 μVp-p. Further, the ECG may be configured with an input impedance of greater than or equal to about 5 MΩ (single ended) and a common mode rejection ratio of greater than or equal to about 89 dB. The ECG bandwidth in a monitoring mode may be between about 0.5 Hz and about 40 Hz and a diagnostic mode may be between about 0.05 Hz and about 75 Hz. In some embodiments, the ECG is configured to detect up to 16 types of arrhythmias based on a five channel ECG.

Strip connector 147 is the panel-mounted connector for receiving the electronic strips used to measure one or more analytes. For example, blood glucose, uric acid, triglycerides, and total cholesterol may be measured. Strip connector 147 is connected to hardware board 140 through a dedicated wired cable.

Now referring to FIG. 6 showing a bottom side of the device. Feet 150, 151 are rubberized mechanical stiffeners attached to a region of the back portion 112 of the housing. Feet 150, 151 provide partial adhesion to a surface on which the device rests to prevent movement of the device while it is being operated. In addition, feet 150, 151 create a space below the device for airflow. Feet 150, 151 prevent the device from contacting any objects lying on the surface on which the device rests.

Now referring to FIG. 7, which shows an interior view of a back portion 112 of one embodiment of a portable diagnostic kit, with the front portion of the device removed. Motherboard SBC 113 performs various functions, such as collecting data, analyzing, and generating results. The results may be in the form of numeric values for each measured patient parameter. A custom-built operating system kernel runs on the SBC to monitor the sub-systems in real-time, smoothen the signals resulting from the sub-systems (e.g., probes, strip module, etc.), and generate results as well as alerts based upon the results. Hardware board 114 processes ECG signals coming from panel mounted ECG port 109, as shown in FIG. 3. In some embodiments, the portable medical diagnostic device combines both a five channel ECG and a twelve channel ECG for optimum diagnosis of heart problems without reduced accuracy or detailed ECG as required.

Signal processing hardware 121 processes signals from analyte tests based on electronic strips. Signal processing hardware 121 and hardware board 114 connect to the motherboard SBC 113 through dedicated wired communications links. Signal processing hardware 121 can sense different types of strips (e.g., glucose B23, uric acid B25, triglyceride B26, or cholesterol B27, as shown in FIG. 8 below) automatically and inform the user of the test that can be performed with the particular detected strip type. Cover 115 for the battery compartment prevents fire and/or arc from the battery from impacting other sections/sub-systems of the device and isolates the battery pack from rest of the device. Mechanical support panel 116 mounts the display 110 (e.g., touchscreen) onto the front portion 108 of the housing. Panel 116 holds the touchscreen display 110 in position on the housing, aligns the display 110, and isolates the display 110 from sensitive electronics in the rear section of the device housed in back portion 112. The back portion 112 of the housing and front portion 108 of the housing are mated to each other mechanically through threaded bosses 117, 118, 119, 120.

Now referring to FIG. 8, which illustrates a rear view of the device. Apertures 122, 123 are for installing the screws through the bosses 117, 118,119 and 120. Air vents 124 are configured for cooling the internal part of the device through airflow. Surface curvatures 125, 126 are present to allow the device to rest on a surface while still displaying the display at an appropriate angle for the user. For example, a display of the device, when resting on a surface, may be angled relative to the surface at about 45 degrees to about 90 degrees, about 60 degrees to about 80 degrees, etc.

In some embodiments, the PDK further includes one or more limit alarms, for example to indicate when one or more parameters or measurements exceed the specifications, indicate a danger scenario, or other improper use. Such limit alarm may be configured to automatically initiate an alert to a healthcare provider or a technician for servicing the device. The alert may be sent via email, phone, or SMS.

The PDK may include a display, for example a touchscreen, interactive interface. It displays, in real-time, a patient's parameters and waveforms. The display is divided into three area: information area 162 (including T7, T8, T9), parameter area 164 (including T1-T6, T11-T17), and waveform area 160 (including T10, T18), as shown in FIG. 9.

The following elements on the display allows the user to interact with the system and perform various tests. Indicators T1, T2, T3, T4, T5, and T6 emit audio, light, or haptics continuously at a rate (e.g., once per second, once per several seconds, once every 30 seconds, once every minute, etc.) to indicate that the test is currently active. Once the test is completed, the indicator stops emitting an alert (e.g., audio, light, haptics, etc.). Indicators T1, T2, T3, T4, T5, and T6 may be virtual or physical buttons, sliders, or other input element.

In some embodiments, the parameters are displayed in different colors for visual differentiation. For example, analyte indicator T1 initiates blood glucose, uric acid, triglycerides, and/or total cholesterol measurement. Depending on the strip inserted, the appropriate test is performed. Strip sub-display T15 displays the analyte levels or details depending on the electronic strip inserted.

BP indicator T2 initiates noninvasive blood pressure measurement. The cuff is automatically inflated to full occlusion and gradually deflated to arrive at correct systolic and diastolic blood pressure values. The values (e.g., diastolic pressure, systolic pressure, etc.) are displayed on screen at BP sub-display T16.

ECG indicator T3 initiates ECG measurement. The leads of the ECG probe are connected to various points on the body. As soon as the probes are connected correctly, ECG waveform is observed on the screen. ECG sub-display T18 displays the ECG waveforms. The heart rate and respiratory rate derived from the ECG waveform is displayed on the screen at ECG sub-display T17.

SpO2 indicator T4 initiates SpO2 measurement(s). Plethysmograph, oxygen saturation, and pulse rate are measured as part of the test. Oxygen saturation percentage and pulse rate are displayed at SpO2 sub-display T13.

Temperature indicator T5 initiates temperature measurement. The probe may be an oral probe, an under the arm type probe, or a contact-based probe (e.g., forehead). The body temperature is displayed on screen at temperature sub-display T14.

Weight indicator T6 initiates body weight measurement(s). The weighing machine is connected to the device via Bluetooth or other wireless technology. The weight value is displayed at weight sub-display T12.

Selection of record input T7 initiates recording of all the parameters and/or results along with a clipping (e.g., 10 seconds) of all the channels of ECG waveform. If the patient profile is not selected/created, it will display an error. The recorded data along with patient profile information is stored on the local nonvolatile memory of the device.

Selection of options input T8 displays different options for the user to select. Some of the options, like connect to a Bluetooth device, synchronize data to cloud server, and/or the like are initiated from this menu, as will be described elsewhere herein.

Profile sub-display T9 displays the selected patient profile. PPG sub-display T10 displays plethysmograph (PPG) waveform live. Time sub-display T11 displays the current time.

To power up or turn on the PDK, a power cable is connected to an external power outlet. Alternatively, an internal, rechargeable battery is used to power the device. A user input (e.g., button, switch, etc.) is depressed on a housing of the device to switch on and off the device. In some embodiments, an initial boot up screen appears. After a few seconds, the PDK home screen appears, as shown in FIG. 9.

A method of initialization and/or authentication of a PDK is shown in FIG. 10. The method starts at block S200. The system is powered on or woken up at block S202 by either user input to select a power button or by tapping or double tapping the display of the device. At block S204, a splash screen is displayed on the display of the device. If the device does wake up at block S206, the display presents the home screen. Additionally, if a user selects home using a user input element at block S208, the home screen is displayed at block S210. A user may then select, using a user input element (e.g., button, slider, haptic input, etc.) one of a plurality of options: system settings at block S121, parameters at block S214, profile at block S216, record at block S218, and options at block S220. At block S222, the system (e.g., server, cloud, etc.) determines whether the PDK is detected by the system (e.g., server, cloud, etc.). If the PDK is not detected by the system (e.g., server, cloud, etc.), the PDK waits for a new connection initiation at block S224 and may optionally return to the home screen at block S226 during the new connection initiation process. If the PDK is detected by the system (e.g., server, cloud, etc.) at block S222, the system (e.g., server, cloud, etc.) connects to the PDK at block S228. If there is a failure to connect, the home screen may be displayed on the PDK at block S230. If the PDK is successfully connected, the PDK checks for device signature or authentication to verify that an authorized user is using the PDK. If there is success, all previous parameters stored in the PDK are cleared from the PDK at block S238. If verification or authorization is not successful, the PDK displays the home screen at block S236. Once the device parameters are cleared, the connection status is displayed as “on” on the screen at block S240 and the home screen is displayed at block S242.

In some embodiments, when a new user or patient is using the PDK, a new patient profile may be created, as first introduced at block S216 of FIG. 10. A method for a new profile creation is shown in FIG. 11. Patient data may be stored in nonvolatile memory of the PDK and uploaded to a server, automatically or on-demand The method starts at block S300. Profile is selected at block S302 to start the new profile creation process. At block S304, a profile create form is displayed on the display of the PDK and a customer ID is received as input. The system determines whether the customer ID exists in a database at block S306. If not, the method proceeds to capturing input fields at block S308. Capturing input fields at block S308 includes: capturing bibliographic data (e.g., name, date of birth, age, mobile number, address, gender, height, etc.) at block S324 and capturing a photo or an image of the patient or user at block S326. If the customer ID does exist at block S310, details from the local database are loaded. At block S312, the method waits for user confirmation of the details loaded at block S310 and/or block S308. If there is no confirmation, the home screen is displayed at block S314. If the details are confirmed at block S312, the method proceeds to block S316 which includes setting the current user as user ID and displaying the user ID details on the display at block S318. Once completed, the home screen is displayed at block S320.

In some embodiments, one or more system settings may be adjusted, as first introduced at block S212 in FIG. 10. A method of adjusting system settings is shown in FIG. 12. When SYS is selected at block S400 by user input, system settings may be adjusted at block S402, any of which, as shown in FIG. 12, may be saved to or updated in backend database B. For example, an audio indication at block S204 of one or more user input elements (e.g., when tests are being run) may be turned on or off or activated or deactivated at block S422. Further, Wi-Fi settings at block S406 may be adjusted or activated/deactivated. Connection to a cloud server may be initiated at block S408, for example by entry of a URL for the server at block S426 and a username and password at block S428, to ensure a secure connection. At block S410, video call configurations for a remote health appointment may be configured, for example by configuring an internet protocol (IP) or IP address at block S430. Various probes and analytics may be calibrated at block S412, for example using one or more test, previous, or actual datasets at block S432. In some embodiments, emergency settings may be adjusted at block S414, for example a call number for emergencies may be input or changed at block S434. In some embodiments, service center settings may be adjusted at block S416, for example a call number for servicing the device may be input or changed at block S436. An idle timeout setting may be input or adjusted at block S418, for example an idle time until timeout may be set at block S438 (e.g., if device is idle for X minutes, timeout and/or lock the device). Lastly, settings may be exited at block S420, identified as ESC or escape.

FIG. 13 shows various options as first shown at block S220 in FIG. 10. FIG. 13, ideally, is performed on the PDK but may, alternatively, be performed on another device (e.g., healthcare provider computing device, etc.) that is in communication with the PDK. FIG. 13 shows various options for setting up a PDK for use by a user, for example a patient. The method starts at block S500 with selection of options at block S502. If a user selects new device at block S510, the display proceeds to display a list of known devices at block S512. If no existing device is selected, the method proceeds to block S514, in which the backend database, server, or cloud is used to capture a new device ID. If an existing device is selected from the list at block S512, the method proceeds to block S516 in which a device connection is initiated. If the connection fails, an error message to displayed at block S518 or any other type of error is indicated, for example audio, visual, or haptic, and the method returns the display to the home screen at block S526. If device connection is successful, the method proceeds to block S520 in which a test communication with the device is run, for example, to ensure that the device can be used for a remote health appointment. The device is then configured at block S522, the device ID is saved to a default device list at block S526, and the display returns to the home screen at block S526. In some embodiments, it may be desired to disconnect the PDK at block S528. In such embodiments, the device ID is selected at block S530. If no device ID is selected, the display returns to the home screen at block S526. If the device ID is selected, the method proceeds to block S532, in which the device confirms the user. If the user is confirmed, the device initiates a hardware disconnect at block S534, updates the display at S536, and updates the device list at block S538 to remove the disconnected device from the list. The display then returns to the home screen at block S526.

In some embodiments of the method of FIG. 13, a selection via a user input element may indicate that synchronization is desired at block S540. In such embodiments, the method proceeds to check a URL setup at block S542. If the URL setup is successful, the method proceeds to block S544 where the device checks for new records on the local database. If no new records are available, the method proceeds to the home screen at block S526. If there are new records, the method proceeds to block S546 in which the device connects to a server, for example a cloud server. If the connection is unsuccessful, the home screen is displayed at block S526. If the connection is successful, the method proceeds to block S548, in which the record is uploaded to the server. If the upload fails, the home screen is displayed at block S526. If the upload is successful, the method queries the device for more new records at block S550. If there are more records, the method loops back to block S548. If there are no new records, the method continues to block S552 to indicate a complete upload and unmark the records in the database at block S554. The display then returns to the home screen at block S526. In some embodiments, resynchronization is required at block S504. In such embodiments, all records that were not sent are marked and then the method returns to block S508 to check the URL set-up at block S542.

In some embodiments, clean-up is selected at block S556. The clean-up is confirmed at block S558, for example via user input or user selection of a user input element to confirm, then the device erases all or a subset of the databases and returns to home at block S526. In some embodiments, back-up is required or selected at block S562. User confirms at block S564, via user input, a copy of the database is created at block S566, all records are copied at block S568, a drive is selected for the back-up at block S570, and a back-up file is created at block S572. All the files are encrypted at block S574 and saved to the selected drive at block S576. The method returns to home at block S526. In some embodiments of data back-up or file transmission, HIPPA compliant encryption is used, for example end-to-end encryption. End-to-end encryption is a means of transferring encrypted data such that only the sender and intended recipient can view or access the transferred data. This is distinct from other means of data transfer wherein encrypted data is temporarily stored on an intermediary server. In some embodiments, full disc encryption, such that any and all data on the PDK are encrypted.

FIG. 14 shows a method of recording an ECG measurement using a PDK. In some embodiments of the method of FIG. 14, a display of the PDK may show various recommendations or warnings, for example, make sure the patient is lying on his/her back before connecting electrodes, leads, and cable to the patient; patient must be stationery during the measurement, correct placement of electrodes or leads on the patient; etc. Once the electrodes and leads are connected, the method of FIG. 14 starts at block S600 with user selection of REC for recording the ECG waveforms at block S602. The PDK queries whether a user profile has been selected, and if no profile has been selected, the display shows an error at block S616 indicating that no profile was selected. If a user profile was selected, the device starts a ten second timer at block S604 and collects ECG data or samples at block S606. If the ten second timer has not timed out at block S6008, the method continues at block S606 to collect additional ECG data. If the 10 second timer expires, the system collects active parameter results (i.e., real-time measurement of ECG signal data) at block S610. The results of the ECG are stored in a local databased on the PDK at block S612 and marked for upload to a server at block S614 (e.g., marking may include encrypting, associating with one or more patient files or profile information, etc.). The PDK displays the ECG waveform, beats per minute (i.e., heart rate), and/or the respiratory rate on the display, for example simultaneously during a remote health appointment. The ECG results may be transmitted substantially simultaneously, in real-time to a remote computing device so that the healthcare provider can view the results during the remote health appointment.

Any of the methods of FIGS. 15-20 and/or any health parameter analysis may be performed locally on the PDK using edge computing methods, such that sensitive patient data is not transmitted to the cloud or a remote server for processing and/or analysis. Such data, after processing, encryption, and/or the like may be transmitted to the cloud, server, and/or remote computing device for storage and/or viewing by a healthcare provider during a remote appointment. Further, any of the methods of FIGS. 15-20 and/or any health parameter analysis may be performed using machine learning and/or artificial intelligence methods locally on the PDK using edge computing methods, for example using training datasets based on anonymized patient data including raw signals for one or more health parameters or analyzed health parameters. Further, one or more data backups, alerts, recommendations, and/or messages may be transmitted to the cloud, server, or a remote computing device.

FIG. 15 shows a method of measuring blood pressure using a PDK. In some embodiments of the method of FIG. 15, a display of the PDK may show various recommendations or warnings, for example, make sure you empty the cuff until there is no residual air inside it to increase accuracy; wrap the cuff around patient's upper arm evenly to appropriate tightness; select appropriate cuff according to the age of the patient (e.g., width should be ⅔ of the length of the upper arm; inflation part should be long enough to permit wrapping 50-80% of the limb concerned); position the cuff in such a way that the word ARTERY on the cuff is at a location where the clearest pulsation of brachial artery is observed; tighten the cuff such that one finger can be inserted inside the cuff; the lower end of the cuff should be 2 cm above the elbow joint; etc. The method of FIG. 15 starts at block S700 with user selection of non-invasive blood pressure (NIBP) at block 702. The display may show a recommendation to a user to relax and/or get comfortable. At block S704, the method includes displaying a picture for cuff positioning on an arm of a patient in a seated position. At block S706, the system checks a pulse rate of the patient using an SpO2 probe. If the pulse rate is not normal, the system continues to check the pulse rate at block S706. If the pulse rate is normal, the method proceeds to block S708, in which cuff inflation is initiated. Inflation and deflation pressure values are monitored for correct operation at block S712. If pressure values are approved, the method proceeds to deflate the cuff completely at block S714 and returns to start at block S700. Further, the method waits for the results at block S716 and validates the results at block S718. If the results are not valid, an error message may be displayed, and the method may return to start at block S700. If the results are valid, the display of the PDK displays the systole and diastole values at block S720, displays a derived heart rate value at block S724, and deflates the cuff completely at block S726 to restart the method at block S700. The PDK displays the blood pressure measurement, pulse rate, and/or respiration rate on the display, for example simultaneously during a remote health appointment. The blood pressure results may be transmitted substantially simultaneously, in real-time to a remote computing device so that the healthcare provider can view the results during the remote health appointment.

FIG. 16 shows a method of measuring one or more analytes (e.g., glucose, uric acid, triglyceride, cholesterol, viral antigens, anti-viral antibodies, etc.) using a PDK. The method of FIG. 16 starts at block S800 with user selection of a type of analyte measurement at block S802. The method continues to initiate a timer, for example five minutes, to determine whether a strip is inserted at block S804. If the strip insertion is not detected at block S804, the timer expires, and the method returns to start at block S800. If the inserted strip is detected at block S804, the method continues to display information or status (e.g., strip inserted, strip detected, etc.) at block S806. The PDK determines whether the strip is known (e.g., expired, previously used, etc.) at block S808 and if it is, the PDK determines the test type at block S810. For example, the PDK may automatically detect a test type based on the detected strip. The strips may be identified based on a barcode, QR code, near-field communication tag or other type of tag, or some other type of mechanical or electronic identifier. Once the test type is detected at block S810, the display of the PDK displays the test strip type at block S812. At block S814, the PDK determines whether the inserted strip is new, and if it is, the PDK waits for the user to deposit a bodily fluid sample on the strip, for example blood urine, interstitial fluid, saliva, buccal swab, nasal aspirate, etc. at block S816. If the PDK times outs at block S826, the method returns to start at block S800. If the sample is detected and the sample analyzed at block S818, the method acquires results from the sample at block S820. The method validates the results at block S822. If the results cannot be validated at block S822, the display of the PDK displays an error at block S828. If the results are validated, the display of the PDK displays a value of the analyte, for example, in mg/dL, nmol/L, etc. In some embodiments, a display of the PDK further displays the value of the analyte with respect to normal or healthy values, a range of values, a past value of the same patient, or a rolling history of past values for the same patient.

In some embodiments, as shown in FIG. 17, a method of parameter selection includes selecting one or more of the following measurements: SpO2, temperature, ECG, and weight. The method starts at block S900 with selection of SpO2 at block S902, temperature at block S928, ECG at block S942, and/or weight at block S944. Starting first with selection of SpO2 at block S902. The method starts by checking the connection to hardware (e.g., the SpO2 probe) at block S918. If no connection is detected, the method returns to start at block S900. If a connection is detected, the method continues to block S904, which includes waiting for first data from the SpO2 probe connected to the patient. If the first data is detected, the method continues to block S908 to initiate a timer and wait for minimum data at block S910. If minimum data is detected, the method continues to block S912 to display a plethysmogram waveform. If minimum data is not detected, the method returns to block S910 to wait for minimum data. Block S908, in which a timer is initiated, also continues to the active parameter user input, for example, by audio or visual indication (beeping or blinking) to show which test is currently active. The display of the PDK proceeds to display the SpO2 value and/or pulse rate. Once the SpO2 value, pulse rate, and/or waveform are displayed, the method proceeds to block S914 in which the method concludes. The method may continue to loop the data at block S920, either based on past data or newly collected data.

If temperature is selected at block S928, the method continues to check whether the hardware (e.g., temperature probe) is connected. If the probe is not connected, the method returns to start at block S900. If the probe is connected, the method continues to block S930 to wait for first data. A timer is activated at block S934, temperature of the patient is read at block S936, the data is displayed on the display of the PDK at block S938, and the user input element (e.g., button, slider, virtual button, etc.) activates (e.g., audio, visual, haptic, etc.) at block S940 to indicate which test is currently active. The method proceeds to stop at block S914 and then the method restarts at block S900. Alternatively, the method may loop back to block S934 to initiate the timer and wait for a temperature read at block S936.

A method for assessing a weight of a patient starts at block S944. The PDK determines whether a scale is connected to the PDK, for example via a wireless connection, at block SS930. If no scale is detected, the method returns to start at block S900. If a scale is detected, the method waits for tare 0.0 kg or 0.0 lb at block S946. If the scale is not at 0.0 kg or 0.0 lb, the method returns to start at block S900. If the scale is zeroed out, the method continues to block S948, which informs the user, for example via a pop-up or other display feature, to stand on the scale. If no minimum weight is detected, the method loops back to block S948. If a minimum weight is detected, the method proceeds to block S950 to wait for stabilization. Once stabilized, a final weight is detected at block S952. If stabilization is not achieved, the method returns to block S948. The final weight is displayed on a display of the PDK at block S954 and then the method returns to start at block S900.

In some embodiments, a connected scale to the PDK may further include body composition assessment capability. In such embodiments, the method of FIG. 19 may be further included. The method of FIG. 19 starts at block S2000. The user selects the test type using a user input element, for example wellness or body composition assessment or BCA at block S2002. As indicated earlier, a user's height may have already been entered during the user profile method. If a user's height has not been previously entered, the method may prompt the user to enter a height. The PDK may then recommend that the user have bare and dry feet at block S2004. If no body impedance is detected at S2006, the method loops back to block S2004. If body impedance is detected, the device determines whether the body impedance is within range at block S2006. The method then proceeds to block S2008 to prompt the user to stay or stand still. Data are collected and collated at block S2010, to make sure there are data coherence. The method then proceeds to compute BCA parameters such as body mass ratio, visceral fat, body fat percent, muscle mass, bone mass, water content, body mass index, body age, protein mass, etc. at block S2012. One or more BCA parameters are displayed on a display of the PDK and then the method ends at block S2016. In some embodiments, a user may toggle between various BCA parameters on the display using a user input element.

A method of recording an ECG at block S942 of FIG. 17 was shown and described in connection with FIG. 14 above. A method of acquiring ECG is now described in connection with FIG. 18. An ECG method starts at block S1000 with waiting for first ECG data at block S1002. The device determines whether the leads were connected correctly at block S1006. If not, the device informs the user via a pop-up on the display or other indicator (e.g., audio, visual, haptic) at block S1004. When the leads are determined to be connected properly, the method returns to block S1002 to wait for minimum data at block S1010. The device computes the waveforms at block S1012 and displays the waveforms on a display of the PDK at block S1014. Further, the display of the PDK is updated to display a heart rate and/or respiratory rate at block S1016. The device may further update an audio or visual indicator frequency to indicate that the measured signal has stabilized and/or the test has ended or completed at block S1018. The method then loops back at S1008, from block S1018 or S1006, to acquire additional ECG data.

Turning now to FIG. 20, which shows a method of collecting one or more health parameters during a remote health appointment. The method includes: providing a portable device configured to, substantially simultaneously, measure one or more health parameters and conduct a remote health appointment at block S1102; optionally receiving a biometric of a patient as input into the portable device at block S1104; optionally verifying the patient of the portable device based on the biometric at block S1106; receiving a user input, on the display of the portable device, to initiate a remote health appointment with a healthcare provider at block S1108; displaying the healthcare provider in a video, in real-time, on the display at block S1110; acquiring one or more health parameters using one or both of: the one or more probes or the strip sensing module of the portable device during the remote health appointment at block S1112; transmitting, in real-time to a remote computing device, the one or more health parameters to the healthcare provider during the remote health appointment at block S1114; and optionally receiving, in real-time on the portable device, a recommendation from the healthcare provider based on the one or more health parameters at block S1116.

As shown in FIG. 20, the method includes providing a portable diagnostic device at block S1102. The portable diagnostic device may include any one or more of the features shown and described in connection with FIG. 1. For example, the portable diagnostic device may include one or more probes and/or a strip sensing module. The portable diagnostic device may include a housing defining one or more apertures or connectors for coupling to the one or more probes. Further, the portable diagnostic device may include a 3G/4G model for initiating a remote health appointment with a healthcare provider (block S1108) while the one or more probes and/or strip sensing module is used to determine one or more health parameters of the patient (at block S1112). The one or more health parameters may, at least in part, be analyzed locally on the portable diagnostic device using edge computing (e.g., via an edge-computing enabled processor) and then transmitted to a remote computing device, for example a server for further analysis and/or a remote computing device of the healthcare provider using the Wi-Fi module (at block S1114). The healthcare provider may then review the one or more health parameters substantially simultaneously while conducting the remote health appointment.

In some embodiments, the portable device may be further configured to receive a recommendation from the healthcare provider based on the one or more health parameters. For example, the recommendation may be received via the Wi-Fi module and/or the 3G/4G modem. The recommendation may include a prescription, a therapy, a follow-up appointment, dietary restrictions, exercise recommendations, additional testing using the portable device or another device, etc.

The systems and methods of the preferred embodiment and variations thereof can be embodied and/or implemented at least in part as a machine configured to receive a computer-readable medium storing computer-readable instructions. The instructions are preferably executed by computer-executable components preferably integrated with the system and one or more portions of the processor on the hardware board, motherboard SBC, and/or computing device. The computer-readable medium can be stored on any suitable computer-readable media such as RAMs, ROMs, flash memory, EEPROMs, optical devices (e.g., CD or DVD), hard drives, floppy drives, or any suitable device. The computer-executable component is preferably a general or application-specific processor, but any suitable dedicated hardware or hardware/firmware combination can alternatively or additionally execute the instructions.

As used in the description and claims, the singular form “a”, “an” and “the” include both singular and plural references unless the context clearly dictates otherwise. For example, the term “probe” may include, and is contemplated to include, a plurality of probes. At times, the claims and disclosure may include terms such as “a plurality,” “one or more,” or “at least one;” however, the absence of such terms is not intended to mean, and should not be interpreted to mean, that a plurality is not conceived.

The term “about” or “approximately,” when used before a numerical designation or range (e.g., to define a length or pressure), indicates approximations which may vary by (+) or (−) 5%, 1% or 0.1%. All numerical ranges provided herein are inclusive of the stated start and end numbers. The term “substantially” indicates mostly (i.e., greater than 50%) or essentially all of a device, substance, or composition.

As used herein, the term “comprising” or “comprises” is intended to mean that the devices, systems, and methods include the recited elements, and may additionally include any other elements. “Consisting essentially of” shall mean that the devices, systems, and methods include the recited elements and exclude other elements of essential significance to the combination for the stated purpose. Thus, a system or method consisting essentially of the elements as defined herein would not exclude other materials, features, or steps that do not materially affect the basic and novel characteristic(s) of the claimed disclosure. “Consisting of” shall mean that the devices, systems, and methods include the recited elements and exclude anything more than a trivial or inconsequential element or step. Embodiments defined by each of these transitional terms are within the scope of this disclosure.

The examples and illustrations included herein show, by way of illustration and not of limitation, specific embodiments in which the subject matter may be practiced. Other embodiments may be utilized and derived therefrom, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is in fact disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A method of collecting one or more health parameters during a remote health appointment, comprising:
providing a portable device configured to, substantially simultaneously, measure one or more health parameters and conduct a remote health appointment, the portable device comprising:
one or more probes configured to measure or collect one or more of: a blood pressure, a temperature, a blood oxygen saturation, an electrocardiogram, an image of an internal anatomical structure, or a combination thereof,
a strip sensing module configured to receive a test strip therein for measuring an analyte,
a wireless communication module for transmitting data to a remote computing device,
a display,
a processor communicatively coupled to the one or more probes, the strip sensing module, the wireless communication module, and the display, and
a computer-readable medium having non-transitory, processor-executable instructions stored thereon, wherein execution of the instructions causes the processor to perform a method comprising:
receiving a biometric of a patient as input into the portable device;
verifying the patient of the portable device based on the biometric;
receiving a user input, on the display of the portable device, to initiate a remote health appointment with a healthcare provider;
displaying the healthcare provider in a video, in real-time, on the display;
acquiring one or more health parameters using one or both of: the one or more probes or the strip sensing module of the portable device during the remote health appointment, wherein the one or more health parameters are selected from the list consisting of: a blood pressure, a temperature, a blood oxygen saturation, an electrocardiogram, a blood glucose level, a uric acid level, a triglyceride level, a cholesterol level, an influenza antigen level, a coronavirus antigen level, an anti-influenza antibody level, an anti-coronavirus antibody level, or a combination thereof;
transmitting, in real-time to a remote computing device, the one or more health parameters to the healthcare provider during the remote health appointment; and
receiving, in real-time on the portable device, a recommendation from the healthcare provider based on the one or more health parameters.

2. The method of claim 1, wherein transmitting, in real-time, further comprises transmitting, in real-time, a patient health history to the healthcare provider during the remote health appointment.

3. The method of claim 1, wherein verifying the biometric of the patient further comprises receiving an image of the patient with an image sensor of the portable device to perform facial recognition of the image of the patient.

4. The method of claim 1, further comprising automatically detecting which of the one or more probes are connected to the portable device before acquiring the one or more health parameters.

5. The method of claim 1, further comprising transmitting a remote user input, from the healthcare provider, from the remote computing device to the portable device to initiate the acquisition of the one or more health parameters using the portable device.

6. The method of claim 1, further comprising transmitting, in real-time, a prescription to a pharmacy for the patient based on the remote health appointment or the acquired one or more health parameters.

7. The method of claim 1, further comprising encrypting the one or more health parameters before transmission.

8. A portable device of collecting one or more health parameters during a remote health appointment, comprising:
one or more probes configured to measure or collect one or more of: a blood pressure, a temperature, a blood oxygen saturation, an electrocardiogram, an image of an internal anatomical structure, or a combination thereof,
a strip sensing module configured to receive a test strip therein for measuring an analyte,
a wireless communication module for transmitting data to a remote computing device,
a display,
a processor communicatively coupled to the one or more probes, the strip sensing module, the wireless communication module, and the display, and
a computer-readable medium having non-transitory, processor-executable instructions stored thereon, wherein execution of the instructions causes the processor to perform a method comprising:
receiving a biometric of a patient as input into the portable device;
verifying the patient of the portable device based on the biometric;
receiving a user input, on the display of the portable device, to initiate a remote health appointment with a healthcare provider;
displaying the healthcare provider in a video, in real-time, on the display;
acquiring one or more health parameters using one or both of: the one or more probes or the strip sensing module of the portable device during the remote health appointment, wherein the one or more health parameters are selected from the list consisting of: a blood pressure, a temperature, a blood oxygen saturation, an electrocardiogram, a blood glucose level, a uric acid level, a triglyceride level, a cholesterol level, an influenza antigen level, a coronavirus antigen level, an anti-influenza antibody level, an anti-coronavirus antibody level, or a combination thereof;
transmitting, in real-time to a remote computing device, the one or more health parameters to the healthcare provider during the remote health appointment; and
receiving, in real-time on the portable device, a recommendation from the healthcare provider based on the one or more health parameters.

9. The portable device of claim 8, further comprising a housing defining one or more ports configured to receive one or more connectors for the one or more probes therein.

10. The portable device of claim 8, wherein the wireless communication module includes one or more of: a Bluetooth module, a Wi-Fi module, or a 3G/4G modem.

11. The portable device of claim 8, wherein the remote computing device comprises a healthcare provider computing device.

12. The portable device of claim 8, wherein the remote computing device comprises a server or a cloud-based database.

13. The portable device of claim 8, further comprising an image sensor configured to take an image of the patient as the biometric of the patient, such that the processor is configured to perform facial recognition on the image of the patient.

14. The portable device of claim 8, further comprising a speaker and microphone to facilitate communication during the remote health appointment.

15. The portable device of claim 8, further comprising a wireless scale communicatively coupled to the processor, wherein the wireless scale is configured to transmit a weight and a body composition assessment of the patient to the processor of the portable device.

16. The method of claim 8, wherein the display is a touch-enabled interactive configurable display.

17. The method of claim 8, wherein the processor is a medical data configurable processor.

18. The method of claim 8, wherein the one or more probes include one or both of an ultrasound probe or a scope for taking the image of the internal anatomical structure.

19. A method of collecting one or more health parameters during a remote health appointment, comprising:
providing a portable device configured to, substantially simultaneously, measure one or more health parameters and conduct a remote health appointment, the portable device comprising:
one or more probes configured to measure or collect one or more of: a vital parameter, a blood parameter, a viral parameter, or an image of an internal anatomical structure, or a combination thereof blood pressure,
a wireless communication module for transmitting data to a remote computing device,
a display,
a processor communicatively coupled to the one or more probes, the strip sensing module, the wireless communication module, and the display, and
a computer-readable medium having non-transitory, processor-executable instructions stored thereon, wherein execution of the instructions causes the processor to perform a method comprising:
receiving a biometric of a patient as input into the portable device;
verifying the patient of the portable device based on the biometric;
receiving a user input, on the display of the portable device, to initiate a remote health appointment with a healthcare provider;
displaying the healthcare provider in a video, in real-time, on the display;
acquiring the one or more health parameters using one or both of: the one or more probes or the strip sensing module of the portable device during the remote health appointment;
transmitting, in real-time to a remote computing device, the one or more health parameters to the healthcare provider during the remote health appointment; and
receiving, in real-time on the portable device, a recommendation from the healthcare provider based on the one or more health parameters.

20. The method of claim 19, wherein acquiring further comprises: receiving one or more signals from the one or more probes, and extracting the one or more health parameters from the one or more signals locally on the portable device using edge computing methods.

21. The method of claim 19, wherein the viral parameter comprises one or more of: an influenza antigen level, a coronavirus antigen level, an anti-influenza antibody level, an anti-coronavirus antibody level, or a combination thereof.

22. The method of claim 19, wherein the vital parameter comprises one or more of: a temperature, a blood oxygen saturation, an electrocardiogram, a weight, a blood pressure, or a combination thereof.

23. The method of claim 19, wherein the blood parameter comprises one or more of: a blood glucose level, a uric acid level, a triglyceride level, a cholesterol level, or a combination thereof.
